(19) United States
(12) Patent Application Publication
(10) Pub. No.: US 2011/0118304 A1
(43) Pub. Date: May 19, 2011

(54) METHOD FOR TREATING OVER-EATING DISORDERS

(75) Inventors: Jens Damsgaard Mikkelsen, Lyngby (DK); Naheed Mirza, Scotland (DK)

(73) Assignee: NeuroSearch A/S, Ballerup (DK)

(21) Appl. No.: 12/743,546

(22) PCT Filed: Nov. 19, 2008
(86) PCT No.: PCT/EP08/65801

§ 371 Date: Jul. 13, 2010

Related U.S. Application Data
(60) Provisional application No. 60/989,318, filed on Nov. 20, 2007.

(30) Foreign Application Priority Data
Nov. 20, 2007 (DK) PA 2007 01641

(51) Int. Cl. A61K 31/439; A61P 25/30

(52) U.S. Cl. 514/304; 514/299

(57) ABSTRACT

A method for treating over-eating disorders, in particular Bulimia nervosa, Binge Eating Disorder (BED), and Compulsive Over-Eating. The invention furthermore relates to novel pharmaceutical compositions for the treatment of over-eating disorders comprising a therapeutically effective amount of a compound of formula I.

### TECHNICAL FIELD

This invention relates to a method for treating over-eating disorders. The invention furthermore relates to novel pharmaceutical compositions for the treatment of over-eating disorders comprising a therapeutically effective amount of a compound of formula I.

### BACKGROUND ART

An over-eating disorder is a complex compulsion to eat. The eating may be excessive (compulsive over-eating); may include normal eating punctuated with episodes of purging; or may include cycles of bingeing and purging. The most prevalent over-eating disorder is Bulimia nervosa. Another widely and rapidly spreading over-eating disorder is compulsive over-eating, also termed Binge Eating Disorder (BED). Eating disorders have severe immediate and long-term health effects and can even be fatal.

Bulimia nervosa is characterized by recurrent episodes of bingeing (eating large quantities of food over short periods of time) followed by attempts to compensate for the excessive caloric intake by such purging behaviors as self-induced vomiting, laxative abuse, severe restrictive dieting or fasting, or excessive exercise. Some bulimics have described their “binge” episodes as a physical high-feeling, losing control, immediate comfort, etc. However, a binge episode also makes the individual feel guilt, shame, embarrassment, and complete failure. Bulimics often try to regain control of themselves and the situation by purging the food and thus making up for their mistake. This leads to feeling famished and empty again, and therefore, in a vicious cycle, another uncontrollable binge, followed by feeling powerless.

Binge Eating Disorder (BED) is similar to bulimia in the recurrent episodes of bingeing; however, binge eaters do not engage in any purging behavior. The binge episodes often take place in secret, when the person is alone, since feelings of shame and disgust often accompany the binge. Binge eaters typically eat very rapidly, hide food, and stuff themselves to the point of feeling sick. A variant of Binge Eating Disorder is Compulsive Over-Eating where sufferers also do not purge food, however they eat regardless of hunger.

WO97/30997 discloses tropane derivatives, their preparation and use as monoamine neurotransmitter, i.e. dopamine, serotonin, and noradrenaline, reuptake inhibitors.

### SUMMARY OF THE INVENTION

It has surprisingly been shown that a compound of formula I in a specified dosage range can be used to treat over-eating disorders.

In its first aspect the invention provides a method for treating over-eating disorders comprising administering to a human a composition comprising a compound of formula I, any of its stereoisomers or any mixture of its stereoisomers, or a pharmaceutically acceptable salt thereof in a therapeutically-effective amount in the range of about 0.1-2 mg API daily.

In another aspect the invention relates to a pharmaceutical composition effective for treating eating disorders in a human, said composition comprising a therapeutically-effective amount in the range of about 0.1-2 mg API daily of a compound of formula I, any of its stereoisomers or any mixture of its stereoisomers, or a pharmaceutically acceptable salt thereof in admixture with one or more pharmaceutically acceptable adjuvants, excipients, carriers and/or diluents.

Other objects of the invention will be apparent to the person skilled in the art from the following detailed description and examples.

### DETAILED DISCLOSURE OF THE INVENTION

In its first aspect the invention provides a method for treating over-eating disorders comprising administering to a human a composition comprising a compound of formula I

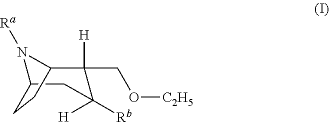

wherein
R^{a} represents hydrogen or alkyl;
R^{b} represents a dihalophenyl group;
any of its stereoisomers or any mixture of its stereoisomers, or a pharmaceutically acceptable salt thereof in a therapeutically-effective amount in the range of about 0.1-2 mg API daily.

The compounds of formula I for use according to the invention are described in WO 97/30997 (NeuroSearch A/S). The compounds may be prepared by conventional methods for chemical synthesis, e.g. those described in WO 97/30997.

In one embodiment of the compound of formula I, R^{a} represents hydrogen or methyl. In a special embodiment, R^{a} represents hydrogen. In a further embodiment, R^{a} represents methyl.

In a further embodiment of the compounds of formula I, R^{b} represents dichlorophenyl. In a special embodiment, R^{b} represents 3,4-dichlorophenyl.

In a still further embodiment, the compound of formula I is tesofensine [(1R,2R,3S,5S)-3-(3,4-dichlorophenyl)-2-(ethoxymethyl)-8-methyl-8-azabicyclo[3.2.1]octane]; or

(1R,2R,3S,5S)-3-(3,4-dichlorophenyl)-2-(ethoxymethyl)-8-azabicyclo[3.2.1]octane; or a pharmaceutically acceptable salt thereof.

In a special embodiment, the compound of formula I is tesofensine or a pharmaceutically acceptable salt thereof. In a further special embodiment, the compound of formula I is the citrate salt of tesofensine.

### DEFINITION OF SUBSTITUENTS

In the context of this invention halo represents fluoro, chloro, bromo or iodo.

In the context of this invention an alkyl group means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of formula I for use according to the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a compound of formula I for use according to the invention include, without limitation, the sodium, the potassium, the calcium, the magnesium, the zinc, the aluminium, the lithium, the choline, the lysinium, and the ammonium salt, and the like, of a compound of formula I for use according to the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the compound of formula I for use according to the invention include examples of suitable prodrugs of the compounds of formula I modified at one or more reactive or derivatizable groups of the parent compound. Of particular interest are compounds modified at a carboxyl group, a hydroxyl group, or an amino group. Examples of suitable derivatives are esters or amides.

The compound of formula I for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of formula I may exist in different stereoisomeric forms—including enantiomers, diastereomers and cis-trans-isomers.

The invention includes all such stereoisomers and any mixtures thereof including racemic mixtures.

### Dosage

The dosage of a compound of formula I is determined as the API (Active Pharmaceutical Ingredient), i.e. calculated as the free base.

In the method according to the invention the compound of formula I, any of its stereoisomers or any mixture of its stereoisomers, or a pharmaceutically acceptable salt thereof is administered to a human in need thereof in a therapeutically-effective amount in the range of about 0.1-2 mg API daily.

The actual dosage of each of the active ingredients depends on the nature and severity of the disease being treated, the exact mode of administration, form of administration and is within the discretion of the physician, and may be varied by titration of the dosage to the particular circumstances of this invention to produce the desired therapeutic effect. However, a daily dosage in the range from about 0.1-2 mg API daily, preferably of from about 0.25-1 mg API daily, especially 0.25, 0.5 or 1.0 mg API daily, is suitable for therapeutic treatments.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

Thus in another aspect the present invention provides a pharmaceutical composition effective for treating eating disorders in a human, said composition comprising a therapeutically-effective amount in the range of about 0.1-2 mg API daily of a compound of formula I

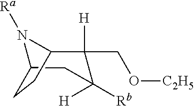

wherein
R^{a} represents hydrogen or alkyl;
R^{b} represents a dihalophenyl group;
any of its stereoisomers or any mixture of its stereoisomers, or a pharmaceutically acceptable salt thereof in admixture with one or more pharmaceutically acceptable adjuvants, excipients, carriers and/or diluents.

The one or more adjuvants, excipients, carriers, buffers, diluents, and/or other customary pharmaceutical auxiliaries must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragée, in powder, or in liquid form, topically such as by inhalation, by patch, enterally, such as by suppository, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

### Biological Activity

The compound of formula I may be used in the method according to the invention for treating over-eating disorders, in particular over-eating disorders selected from the group comprising Bulimia nervosa, Binge Eating Disorder (BED), Compulsive Over-Eating, emotional eating and nighttime eating. Furthermore, the compound of formula I may be used for decreasing appetite, for decreasing an increased appetite, decreasing the desire to eat food or for suppressing hunger.

The activity of the compound of formula I in the method according to the invention will be illustrated by means of the following examples using the compound tesofensine ([(1R,2R,3S,5S)-3-(3,4-dichlorophenyl)-2-(ethoxymethyl)-8-methyl-8-azabicyclo[3.2.1]octane]) as test compound.

### PROTOCOL 1

### Testing Tesofensine in an Eating Behaviour Model

Male Wistar rats with an initial body weight of 300-350 g are housed and maintained on a 12/12 h light-dark schedule (lights on 07:00 h) throughout the experiment. During the handling period (1 wk before the feeding regimen), all rats have ad libitum access to regular laboratory chow pellets and tap water. Throughout the entire experiment all animals remain in their home cages except when daily body weights (BW) are measured between 08:00 and 08:30 h.

Three days prior to the feeding regimen the rats are divided into three groups (n=6 for each) with respect to receiving either low dosis Tesofensine, high dosis Tesofensine, or 0.9% saline (i.e., vehicle) treatments. Accordingly, each group receives a single daily injection (intraperitoneal; IP or Peroral:PO) at 08:30 h for 10 days. All groups are maintained on a food and water restriction schedule consisting of a daily 20 min access to a 0.3 M sucrose solution at 10:00 h, 20 min access to chow at 12:20 h, and a 2 h access to chow and water at 14:00 h. This procedure continues for 7 days and is similar to previously reported scheduled feeding paradigms (N. T. Bello, K. L. Sweigart, J. M. Lakoski, R. Norgren and A. Hajnal, Restricted feeding with scheduled sucrose access results in an upregulation of the rat dopamine transporter, Am. J. Physiol. Regul. Integr. Comp. Physiol. 284 (2003), pp. R1260-R1268). The treatments begin 3 days prior to the feeding regimen not only to ensure appropriate pharmacological effects but also to avoid the initial association between the administration of the drug and the presentation of the palatable sucrose solution.

Chow and water consumption are recorded daily at 08:30 h during the 3-day pretreatment period and sucrose, chow and water intakes are recorded accordingly throughout the 7-day feeding paradigm. Daily cumulative total caloric intake is calculated from the intakes of chow+sucrose and is expressed in kilocalories (Kcal).

All groups are decapitated in a staggered fashion (i.e., to ensure consistency with respect to the feeding regimen) on day 7 at 11:20 h. Trunk blood from each rat is collected into individual EDTA tubes. After removing 20 μl for blood glucose assay, the remainder of blood sample is gently agitated and maintained on ice until centrifugation at 3000 rpm for 10 min. Plasma is then aliquoted into individual microcentrifuge tubes and stored at −80° C. Standard radioimmunoassay kits are used to determine plasma levels of corticosterone, insulin and leptin.

Behavioral data and blood parameters are expressed as mean±S.E. The 20 min food intakes, 2 h food intakes, 2 h water intakes, and daily cumulative caloric intakes are analyzed by ANOVAs with repeated measures. Comparisons between groups are made with one-way ANOVAs for initial and final body weights and blood parameters.

### BRIEF DESCRIPTION OF THE DRAWING

The present invention is further illustrated by reference to the accompanying drawing, in which

FIG. 1 shows the level of food intake over night.

FIG. 2 shows the relation between increasing doses of Tesofensine (s.c.) and the accumulated food intake. **: p<0.01; ***: p<0.001; one-way ANOVA.

### PROTOCOL 2

### Testing the Effect of Tesofensine on Food-Intake in Diet-Induced Obese Rats

These rats were from 3-5 weeks of age only given access to a high-fat diet (#12266B; 31.8% kcal from fat, energy density 4.41 kJ/g, Research Diets, New Jersey). After 17 weeks on high-fat diet, animals were stratified according to body weight. Tesofensine was injected in different doses to male Sprague-Dawley rats. Vehicle (saline), or tesofensine at different doses (from 0,5 mg/kg to 2,0 mg/kg) was administered as a single subcutaneous injection at lights out. The level of food-intake was continuously determined over the next 18 hours in an automated food-delivering system.

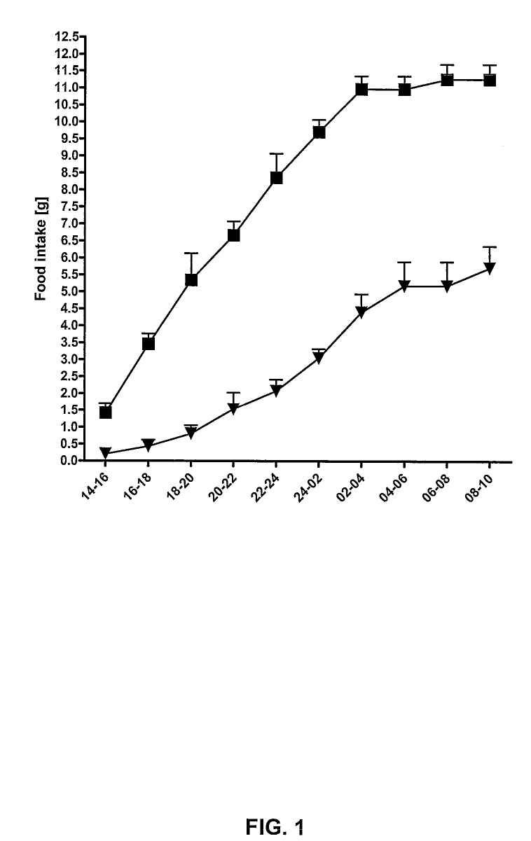

In FIG. 1, the level of food-intake over night is illustrated of two groups of animals; receiving either a single dose of 1,5 mg/kg tesofensine (triangles) or vehicle (squares). It is demonstrated that tesofensine inhibits food intake over night.

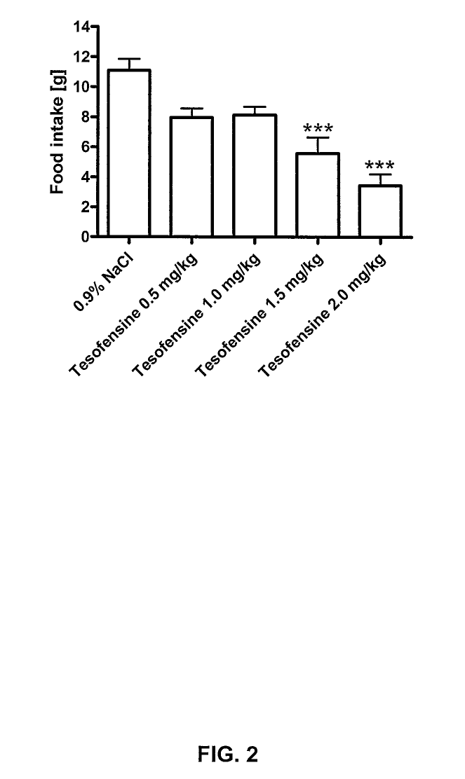

In FIG. 2, a relation between increasing doses of tesofensine and the accumulated food-intake over a 18 h observation is shown.

1. A method for treating over-eating disorders comprising administering to a human a composition comprising a compound of formula I 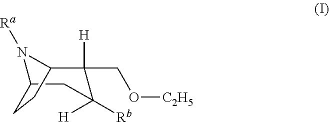
wherein
R^{a} represents hydrogen or alkyl;
R^{b} represents a dihalophenyl group;
any of its stereoisomers or any mixture of its stereoisomers, or a pharmaceutically acceptable salt thereof in a therapeutically-effective amount in the range of about 0.1-2 mg API daily.

2. The method of claim 1 wherein R^{a} represents hydrogen or methyl.

3. The method of claim 1, wherein R^{b} represents 3,4-dichlorophenyl.

4. The method of claim 1 wherein the compound of formula I is tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane; or a pharmaceutically acceptable salt thereof.

5. The method according to claim 1, wherein the daily dosage of the compound of formula I is 0.1-2 mg API daily.

6. The method according to claim 1, wherein the over-eating disorders are selected from the group comprising Bulimia nervosa, Binge Eating Disorder (BED), Compulsive Over-Eating, emotional eating and nighttime eating.

7. The method according to claim 1, wherein the composition is administered orally, intravenously, intravascularly, intraperitoneally, sub-cutaneously, intramuscularly, inhalatively, topically, by patch, or by suppository.

8. A pharmaceutical composition effective for treating over-eating disorders in a human, said composition comprising a therapeutically-effective amount in the range of about 0.1-2 mg API daily of a compound of formula I 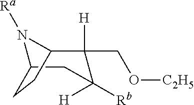
wherein
R^{a} represents hydrogen or alkyl;
R^{b} represents a dihalophenyl group;
any of its stereoisomers or any mixture of its stereoisomers, or a pharmaceutically acceptable salt thereof in admixture with one or more pharmaceutically acceptable adjuvants, excipients, carriers and/or diluents.

9. The composition according to claim 8, wherein R^{a} represents hydrogen or methyl.

10. The composition according to claim 8, wherein R^{b} represents 3,4-dichlorophenyl.

11. The composition according to claim 8, wherein the compound of formula I is tesofensine [(1R,2R,3S,5S)-3-(3,4-dichlorophenyl)-2-(ethoxymethyl)-8-methyl-8-azabicyclo[3.2.1]octane]; or
(1R,2R,3S,5S)-3-(3,4-dichlorophenyl)-2-(ethoxymethyl)-8-azabicyclo[3.2.1]octane; or
a pharmaceutically acceptable salt thereof.

12. The composition according to claim 8, wherein the daily dosage of the compound of formula I is about 0.25-1 mg API daily.

13. The composition according to claim 8, wherein the over-eating disorders are selected from the group comprising Bulimia nervosa, Binge Eating Disorder (BED), Compulsive Over-Eating, emotional eating and nighttime eating.

14. The composition according to claim 8, wherein the composition is administered orally, intravenously, intravascularly, intraperitoneally, sub-cutaneously, intramuscularly, inhalatively, topically, by patch, or by suppository.

15. (canceled)
